(19) United States
(12) Patent Application Publication
(10) Pub. No.: US 2024/0029871 A1
(43) Pub. Date: Jan. 25, 2024

(54) RESIDENTIAL ROOM DETERMINATION APPARATUS, RESIDENTIAL ROOM DETERMINATION METHOD, AND CONTROL PROGRAM

(71) Applicant: KONICA MINOLTA, INC., Tokyo (JP)

(72) Inventor: Tatsuya NAKANO, Sagamihara-shi (JP)

(73) Assignee: KONICA MINOLTA, INC., Tokyo (JP)

(21) Appl. No.: 18/337,254

(22) Filed: Jun. 19, 2023

(30) Foreign Application Priority Data
Jul. 25, 2022 (JP) 2022-117857

(51) Int. Cl. G16H 40/20

(52) U.S. Cl. CPC G16H 40/20

(57) ABSTRACT

An information processing apparatus includes a hardware processor that: acquires care receiver information about a care receiver scheduled to enter a care facility, and room information about each of a plurality of rooms in the care facility; and determines a residential room to be allocated to the care receiver from among the plurality of rooms, based on the care receiver information and the room information.

### CROSS-REFERENCE TO RELATED APPLICATION

Japanese patent application No. 2022-117857 filed on Jul. 25, 2022, including description, claims, drawings, and abstract the entire disclosure is incorporated herein by reference in its entirety.

### BACKGROUND

### Technological Field

The present invention relates to a residential room determination apparatus, a residential room determination method, and a control program.

### Description of the Related Art

In Japan, an increase in longevity has become remarkable by virtue of improvements in a standard of living, a hygienic environment, a standard of medical care, and others associated with the postwar high economic growth. The increase in longevity is combined with a decline in birthrate to result in a graying society where the percentage of elderly people is high. It is assumed in this graying society that the number of care receivers who need care, support, and the like owing to disease, injury, aging, and the like increases.

In the field of care services, various systems have been introduced for supporting caregivers' duties for care receivers. For example, JP 2005-050202 A discloses an example of such a system. There are various care services. For example, some care facilities admit care receivers for a short stay purpose.

### SUMMARY

In a care facility that provides such a care service, it is desirable to lighten a burden on a staff or the like involved in, for example, nursing care, as much as possible.

The present invention has been made in view of the foregoing circumstance. An object of the present invention is to provide a residential room determination apparatus, a residential room determination method, and a control program that are capable of lightening a burden on a staff or the like of a care facility.

The foregoing object of the present invention is attained by the following solution.

A residential room determination apparatus including a hardware processor that: acquires care receiver information about a care receiver scheduled to enter a care facility, and room information about each of a plurality of rooms in the care facility; and determines a residential room to be allocated to the care receiver from among the plurality of rooms, based on the care receiver information and the room information.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a diagram illustrating a general schematic configuration of a system to which a residential room determination apparatus according to an embodiment of the present invention is applied;

FIG. 2 is a block diagram illustrating a schematic configuration of a user terminal illustrated in FIG. 1;

FIG. 3 is a block diagram illustrating a schematic configuration of an information processing apparatus illustrated in FIG. 1;

FIG. 4 is a block diagram illustrating a functional configuration of the information processing apparatus illustrated in FIG. 3;

FIG. 5 is a diagram illustrating exemplary care receiver information acquired by an acquisition unit illustrated in FIG. 4;

FIG. 6 is a diagram illustrating an exemplary arrangement of some rooms in a care facility;

FIG. 7 is a diagram illustrating exemplary room information about each of the rooms in the care facility illustrated in FIG. 6;

FIG. 8 is a diagram illustrating the necessity or unnecessity of application as to each of a plurality of exemplary rules, and exemplary priorities given to the rules, the rules and priorities being accepted by an accepting unit illustrated in FIG. 4; and

FIG. 9 is a flowchart illustrating procedures of processing executed by the information processing apparatus illustrated in FIG. 1.

### DETAILED DESCRIPTION OF EMBODIMENTS

Hereinafter, one or more embodiments of the present invention will be described with reference to the drawings. However, the scope of the invention is not limited to the disclosed embodiments.

An embodiment of the present invention will be described below with reference to the accompanying drawings. In the drawings, the same components are denoted by the same reference sign. In the embodiment and the drawings, a redundant description is omitted. In the drawings, some dimensional ratios are exaggerated for convenience of the illustration and are different from actual ones.

### <Configuration of System>

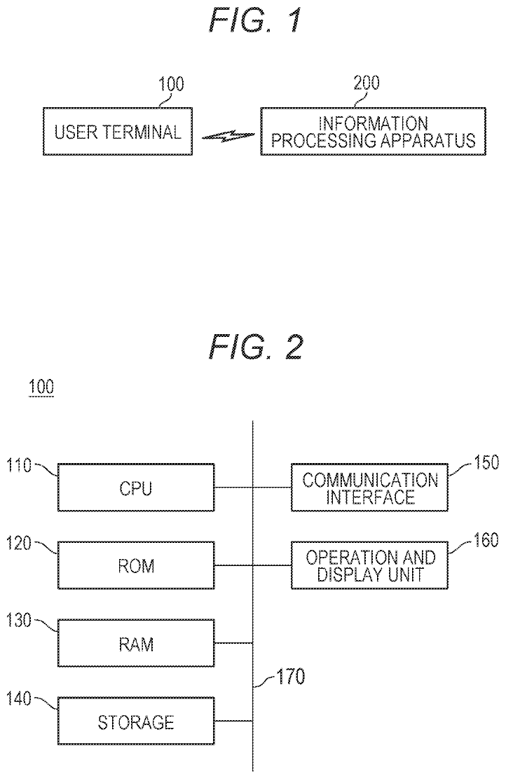

FIG. 1 is a diagram illustrating a general schematic configuration of a system to which a residential room determination apparatus according to an embodiment of the present invention is applied.

As illustrated in FIG. 1, the system includes, for example, a user terminal 100 and an information processing apparatus 200. The user terminal 100 and the information processing apparatus 200 are configured to be connectable to each other via a network. The system is used in, for example, a care facility that admits a care receiver for a short stay purpose.

For example, a care receiver is admitted to make a short stay in a care facility as follows. First, a care receiver himself/herself or his/her family files an application for certification as a care receiver with a municipality. In response to this application, the municipality examines the care receiver's conditions, and then notifies the care receiver or the like of a result of the examination. In the following, the term “care receiver or the like” refers to a care receiver himself/herself or his/her family. The care receiver or the like who has received certification as a care receiver from the municipality consults a care support office or the like on a short stay at a care facility. In response to the consultation, the care support office prepares a document for application, and then submits the document to the care facility. The care facility which has received the document confirms the details of the document while consulting the care support office or the like. Thereafter, the care facility prepares, for example, a reservation list for a short stay once a month while confirming a state of each room in the care facility. The care facility then makes a decision whether to admit the care receiver for a short stay purpose. The care facility notifies the care support office or the like of a result of the decision whether to admit the care receiver. The care facility prepares a list of the care receiver's conditions and a schedule as to when the care receiver enters the care facility and when the care receiver leaves the care facility, until the day at which the care receiver will enter the care facility for a short stay purpose. A staff of the care facility shares the information about the care receiver.

The system illustrated in FIG. 1 is used, for example, at the time when the care facility, which has received the document for application from the care support office or the like, prepares the reservation list for deciding whether to admit the care receiver for a short stay purpose. The system is configured to determine a room for a care receiver scheduled to enter the care facility. In the following, a room for a care receiver is referred to as a residential room.

### (User Terminal 100)

The user terminal 100 is a terminal to be used by a user. Examples of the user include an administrator of the system, and a proprietor, a manager, and a staff or the like of a care facility. The user terminal 100 is, for example, an information processing terminal such as a desktop personal computer (PC), a notebook-sized PC, a tablet PC, or a smartphone.

FIG. 2 is a block diagram illustrating a schematic configuration of the user terminal.

As illustrated in FIG. 2, the user terminal 100 includes a CPU 110, a ROM 120, a RAM 130, a storage 140, a communication interface 150, and an operation and display unit 160. These components are connected to each other via a bus 170 in a communicable manner. The CPU is an abbreviation for “central processing unit”. The ROM is an abbreviation for “read only memory”. The RAM is an abbreviation for “random access memory”.

The CPU 110 controls the foregoing components or executes various kinds of computation processing, in accordance with a program recorded on the ROM 120 or the storage 140.

The ROM 120 stores various kinds of programs or various kinds of data.

The RAM 130 serves as a working area and temporarily stores a program or data.

The storage 140 stores various kinds of data or various kinds of programs including an operating system. For example, an application is installed in the storage 140. The application has, for example, a function of exchanging various kinds of information with another apparatus, such as the information processing apparatus 200, in response to a user's input. The application also has a function of outputting acquired information to the operation and display unit 160.

The communication interface 150 is an interface for communications with another apparatus. Examples of the communication interface 150 to be used herein include communication interfaces conforming to various wired or wireless standards.

The operation and display unit 160 is, for example, a touch screen display. The operation and display unit 160 displays various kinds of information, and receives various user's inputs.

### (Information Processing Apparatus 200)

The information processing apparatus 200 is, for example, a computer such as a server or a PC. The information processing apparatus 200 functions as a residential room determination apparatus in the present embodiment. The information processing apparatus 200 may be practicable using a plurality of apparatuses. The information processing apparatus 200 may alternatively be practicable as, for example, a cloud server in a virtual manner, using a plurality of servers.

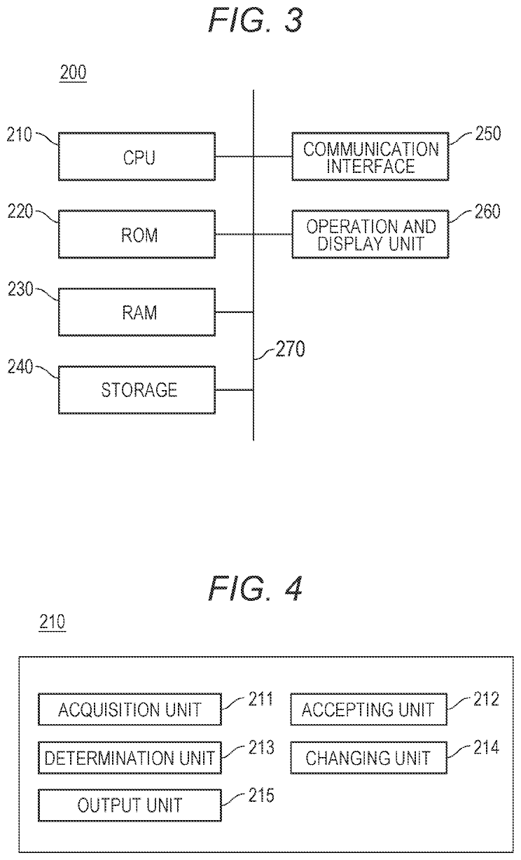

FIG. 3 is a block diagram illustrating a schematic configuration of the information processing apparatus.

As illustrated in FIG. 3, the information processing apparatus 200 includes a CPU 210, a ROM 220, a RAM 230, a storage 240, a communication interface 250, and an operation and display unit 260. These components are connected to each other via a bus 270 in a communicable manner.

These components, that is, the CPU 210, ROM 220, RAM 230, storage 240, communication interface 250, and operation and display unit 260, are similar in function to the corresponding components of the user terminal 100. Therefore, the redundant description is omitted.

The CPU 210 is practicable using a hardware processor to function as an acquisition unit, an accepting unit, a determination unit, a changing unit, and an output unit in the present embodiment. The functions, to be achieved by the CPU 210, of the information processing apparatus 200 will be described in detail later.

A program is installed in the storage 240, and the CPU 210 executes processing in accordance with this program. The storage 240 stores, as an internal database, room information about each of the plurality of rooms in the care facility. The storage 240 also stores care receiver information about a care receiver scheduled to enter the care facility. The storage 240 may store, for example, information about a rule for allocating a suitable room as a residential room to each care receiver scheduled to enter the care facility, and a priority given to the rule. The care receiver information, the room information, and the information about the rule and its priority will be described in detail later. In the present embodiment, the storage 240 is a specific example of a storage unit according to the present invention.

The system may include a constituent element in addition to the foregoing constituent elements. Conversely, the system does not necessarily include one or more constituent elements among the foregoing constituent elements. As to each of the constituent elements, the number is not limited to one and may be more than one.

### <Functions of Information Processing Apparatus 200>

FIG. 4 is a block diagram illustrating a functional configuration of the information processing apparatus.

In the information processing apparatus 200, the CPU 210 reads a program from the storage 240, and executes processing in accordance with the program. The information processing apparatus 200 thus functions as an acquisition unit 211, an accepting unit 212, a determination unit 213, a changing unit 214, and an output unit 215.

The acquisition unit 211 acquires care receiver information about a care receiver scheduled to enter the care facility. The care receiver is, for example, a care receiver scheduled to enter the care facility for a short stay purpose. The care receiver enters the care facility for 30 days at maximum. The acquisition unit 211 acquires the care receiver information from, for example, the storage 240.

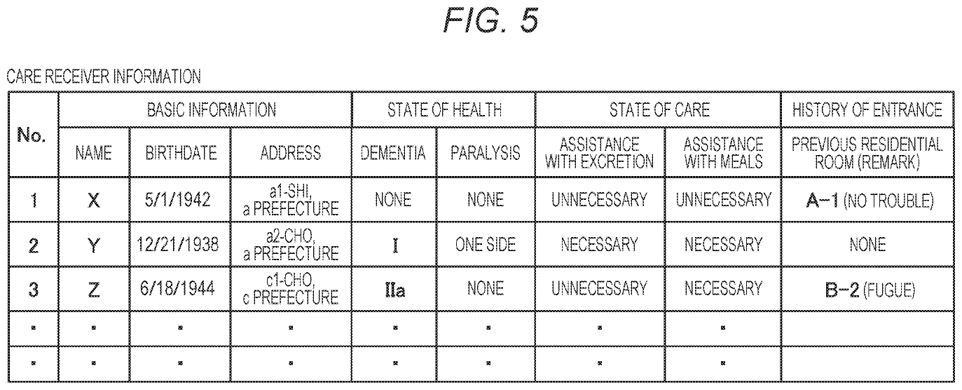

FIG. 5 illustrates exemplary care receiver information. The care receiver information includes, for example, basic information, information about a state of health, information about a state of care, and information about a history of entrance to the care facility, for each care receiver.

The basic information is, for example, information about a name, a gender, a birthdate, an address, and the like for each care receiver. The information about the state of health includes, for example, information about symptoms of dementia, paralysis, and the like for each care receiver. The information about the state of health also includes, for example, information about the presence or absence, a level, and the like of the symptoms. The information about the state of care includes, for example, information about assistance with walking, excretion, meals, and the like for each care receiver. The information about the state of care also includes, for example, information about the necessity or unnecessity, a level, and the like of each assistance. The information about the history of entrance to the care facility include, for example, information about a room used as a residential room at past entrance to the care facility, for each care receiver. The information about the history of entrance to the care facility may include information about the duration of a past stay, the fact of trouble during the past stay, the fact of fugue during the past stay, and the like.

The staff or the like of the care facility inputs the basic information, the information about the state of health, and the information about the state of care for each care receiver to the information processing apparatus 200 with the user terminal 100. The information thus input is stored in the storage 240. For example, the staff or the like of the care facility inputs the information, based on the document for application received from the care support office or the like. The staff or the like of the care facility also inputs the information about the history of entrance to the care facility for each care receiver to the information processing apparatus 200 with the user terminal 100. The information about the history of entrance to the care facility is stored in the storage 240. For example, the staff or the like of the care facility inputs the information about the history of entrance to the care facility, based on a care record or the like in the care facility. The staff or the like of the care facility may input the care receiver information to the information processing apparatus 200 through the operation and display unit 260. The care receiver information may alternatively be acquired from another apparatus (e.g., an external server).

Preferably, the care receiver information includes at least one of the basic information, the information about the state of health, the information about the state of care, or the information about the history of entrance to the care facility, for each care receiver. More preferably, the care receiver information includes all of the basic information, the information about the state of health, the information about the state of care, and the information about the history of entrance to the care facility, for each care receiver. It is thus possible to determine a room more suitable for a care receiver, as a residential room for the care receiver.

The acquisition unit 211 further acquires room information about each of the plurality of rooms in the care facility. The acquisition unit 211 acquires, for example, room information about each room usable as a residential room. The acquisition unit 211 acquires the room information from, for example, the storage 240.

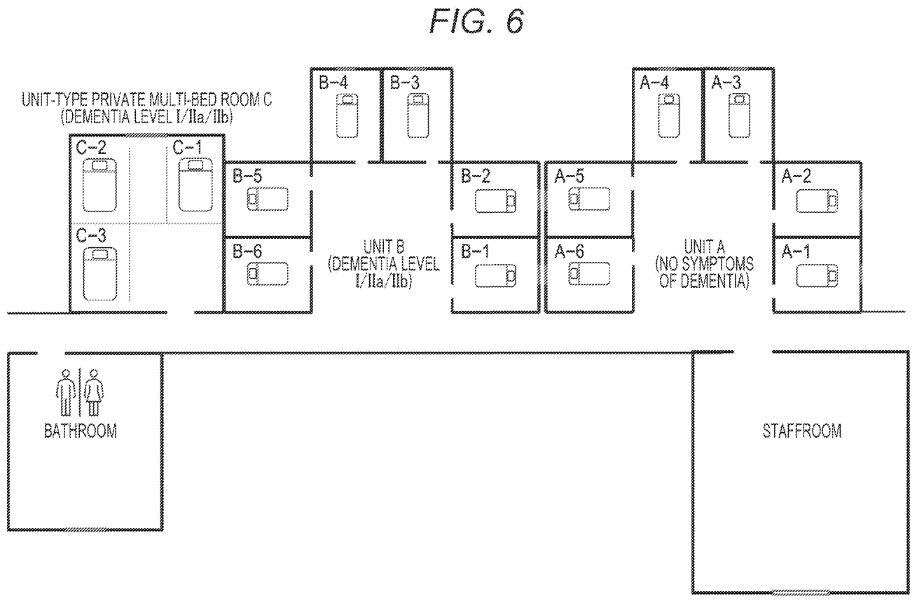

FIG. 6 illustrates an exemplary arrangement of some of the rooms in the care facility. A part of the care facility includes a unit A constituted of six private rooms, a unit B constituted of six private rooms, and a unit-type private multi-bed room C having three beds. A staffroom is adjacent to the unit A, and a bathroom is adjacent to the unit-type private multi-bed room C. The unit A admits a care receiver with no symptoms of dementia. The unit B and the unit-type private multi-bed room C each admit a care receiver suffering from dementia at Level I, IIa, or IIb.

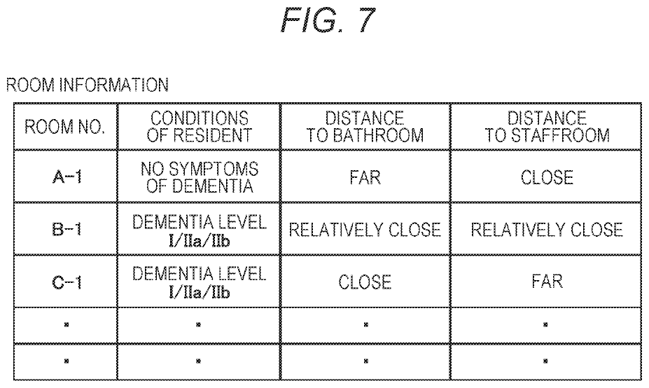

FIG. 7 illustrates exemplary room information about each of the rooms in the care facility illustrated in FIG. 6. The room information includes, for example, identification information such as a room number of each room, information about a location of each room, information about conditions of a resident of each room, and the like. The information about the location of each room includes, for example, information about a distance between each room and the bathroom, a distance between each room and the staffroom, a distance between each room and a place to leave care equipment and the like, and the like. The information about the conditions of the resident of each room includes, for example, information about whether a care receiver who is able to use each room suffers from dementia, a level of dementia, and the like. The room information may include information about vacancy of each room. The room information may also include information about whether equipment such as care equipment is installed in each room.

Preferably, the room information includes at least one of the identification information on each room, the information about the location of each room, or the information about the conditions of the resident of each room. More preferably, the room information includes all of the identification information on each room, the information about the location of each room, and the information about the conditions of the resident of each room. It is thus possible to determine a room more suitable for a care receiver, as a residential room for the care receiver.

The accepting unit 212 accepts user's inputs about a plurality of rules for allocating a suitable room to each care receiver scheduled to enter the care facility, and priorities given to the rules. As will be described in detail later, the determination unit 213 applies the rules, associates the care receiver information with the room information, and determines residential rooms for the care receivers scheduled to enter the care facility.

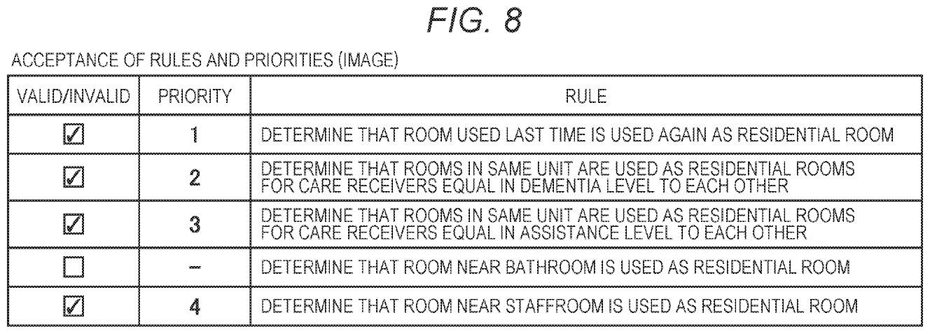

FIG. 8 illustrates exemplary rules and their priorities accepted from the user. For example, a plurality of rules are previously stored in the storage 240 or the like. The user inputs, to the information processing apparatus 200, the necessity or unnecessity of application as to each of the plurality of rules, and a priority given to an applied one of the rules. The rules include, for example, “determine that a room used last time is used again as a residential room”, “determine that rooms in the same unit are used as residential rooms for care receivers equal in dementia level to each other”, “determine that rooms in the same unit are used as residential rooms for care receivers equal in assistance level to each other”, and the like. The rules also include “determine that a room near a bathroom is used as a residential room”, “determine that a room near a staffroom is used as a residential room”, and the like.

The rules, “determine that a room used last time is used again as a residential room”, “determine that rooms in the same unit are used as residential rooms for care receivers equal in dementia level to each other”, “determine that rooms in the same unit are used as residential rooms for care receivers equal in assistance level to each other”, and “determine that a room near a staffroom is used as a residential room”, are applied in the example illustrated in FIG. 8. The highest priority is given to the rule, “determine that a room used last time is used again as a residential room”. The second highest priority, the third highest priority, and the lowest priority are respectively given to the rule, “determine that rooms in the same unit are used as residential rooms for care receivers equal in dementia level to each other”, the rule, “determine that rooms in the same unit are used as residential rooms for care receivers equal in assistance level to each other”, and the rule, “determine that a room near a staffroom is used as a residential room”. The accepting unit 212 may accept a change in the rules stored previously, from the user. The accepting unit 212 may alternatively accept establishment of a new rule.

The determination unit 213 determines a residential room to be allocated to each care receiver, from among the plurality of rooms in the care facility, based on the care receiver information and room information acquired by the acquisition unit 211. In this case, the determination unit 213 identifies a room suitable for each care receiver, in accordance with the plurality of rules and their priorities accepted by the accepting unit 212 from the user. Thereafter, the determination unit 213 determines that each room thus identified is used as a residential room for the corresponding care receiver. Specifically, the determination unit 213 applies the rules in descending order of the priorities, and associates the care receiver information and the room information with each other. The determination unit 213 thus identifies a room suitable for each care receiver. The plurality of rooms in the care facility are, for example, rooms A-1, A-2, . . . , and C-3.

For example, the determination unit 213 applies the rule, “determine that a room used last time is used again as a residential room”. In this case, the determination unit 213 associates the information about the history of entrance to the care facility, the information being included in the care receiver information, with the identification information on each room, the identification information being included in the room information. The determination unit 213 may apply the rule, “determine that rooms in the same unit are used as residential rooms for care receivers equal in dementia level to each other”. In this case, the determination unit 213 associates, for example, the information about the state of health, the information being included in the care receiver information, with the information about the conditions of a resident of each room, the information being included in the room information. The determination unit 213 may apply the rule, “determine that rooms in the same unit are used as residential rooms for care receivers equal in assistance level to each other”. In this case, the determination unit 213 associates, for example, the information about the state of care, the information being included in the care receiver information, with the information about the location of each room, the information being included in the room information. The determination unit 213 may apply the rule, “determine that a room near a bathroom is used as a residential room”. In this case, the determination unit 213 associates the information about the state of care, the information being included in the care receiver information, with the information about the location of each room, the information being included in the room information. The determination unit 213 may apply the rule, “determine that a room near a staffroom is used as a residential room”. In this case, the determination unit 213 associates, for example, the information about the state of health, the information being included in the care receiver information, and the information about the state of care, the information being included in the care receiver information, with the information about the location of each room, the information being included in the room information.

The following description is given with reference to FIG. 5. It is assumed that the highest priority is given to the rule, “determine that a room used last time is used again as a residential room”, and the room A-1 is vacant. In this case, the determination unit 213 determines that the room A-1 used last time is used as a residential room for a care receiver X. If the room A-1 is not usable, the determination unit 213 applies the rule, “determine that rooms in the same unit are used as residential rooms for care receivers equal in dementia level to each other” to which the second highest priority is given. The determination unit 213 then determines that a vacant room in the unit A capable of admitting a care receiver with no symptoms of dementia is used as a residential room for the care receiver X. The following description is given with reference to FIGS. 6 and 7. The vacant room in the unit A capable of admitting the care receiver X is, for example, the room A-2.

The changing unit 214 changes the room for the care receiver, determined as the residential room by the determination unit 213, to a different room. The room for the care receiver determined as the residential room by the determination unit 213 is, for example, a first room. The different room is, for example, a second room. The changing unit 214 changes the residential room for the care receiver to the different room, based on, for example, the plurality of rules and their priorities accepted by the accepting unit 212. For example, the determination unit 213 identifies the first room for a different care receiver by applying one of the rules to which the higher priority is given. At this time, the changing unit 214 changes, to the second room, the residential room for the care receiver to which the first room has already been allocated as the residential room. The following describes an exemplary case where the changing unit 214 changes a residential room for a care receiver.

For example, for a care receiver whose application for entrance has been received first, the determination unit 213 identifies the room B-2 as a room conforming to the rule to which the second highest priority is given. The determination unit 213 then determines that the room B-2 is used as a residential room for the care receiver. The care receiver whose application for entrance has been received first is, for example, the care receiver Y. The rule to which the second highest priority is given is, for example, the rule, “determine that rooms in the same unit are used as residential rooms for care receivers equal in dementia level to each other”. For a care receiver whose application for entrance has been received later, next, the determination unit 213 applies the rule to which the highest priority is given, and identifies the room B-2. At this time, the changing unit 214 changes the residential room for the care receiver whose application for entrance has been received first, from the room B-2 to the room B-3. The care receiver whose application for entrance has been received later is, for example, a care receiver Z. The rule to which the highest priority is given is, for example, the rule, “determine that a room used last time is used again as a residential room”. The care receiver whose application for entrance has been received first is, for example, the care receiver Y.

The changing unit 214 changes, to a different room, the residential room for the care receiver determined by the determination unit 213, based on a user's instruction. The changing unit 214, when receiving no instruction from the user, maintains the residential room for the care receiver determined by the determination unit 213 without changing the residential room for the care receiver determined by the determination unit 213. The residential rooms for the care receivers are thus fixed in the order of, for example, arrival of the applications for entrance to the care facility.

The output unit 215 outputs the information about the residential room for each care receiver determined by the determination unit 213. The output unit 215 outputs, for example, the room number of the room determined as the residential room and the information (e.g., the name) about the care receiver scheduled to enter the room, in association with each other. The information about the care receiver is, for example, the name and the like of the care receiver. The output unit 215 outputs, for example, the information about the residential room for the care receiver in such a manner that the operation and display unit 260 displays the information. The output unit 215 may output the information about the residential room for the care receiver in such a manner that the operation and display unit 160 of the user terminal 100 displays the information.

### <Processing by Information Processing Apparatus 200>

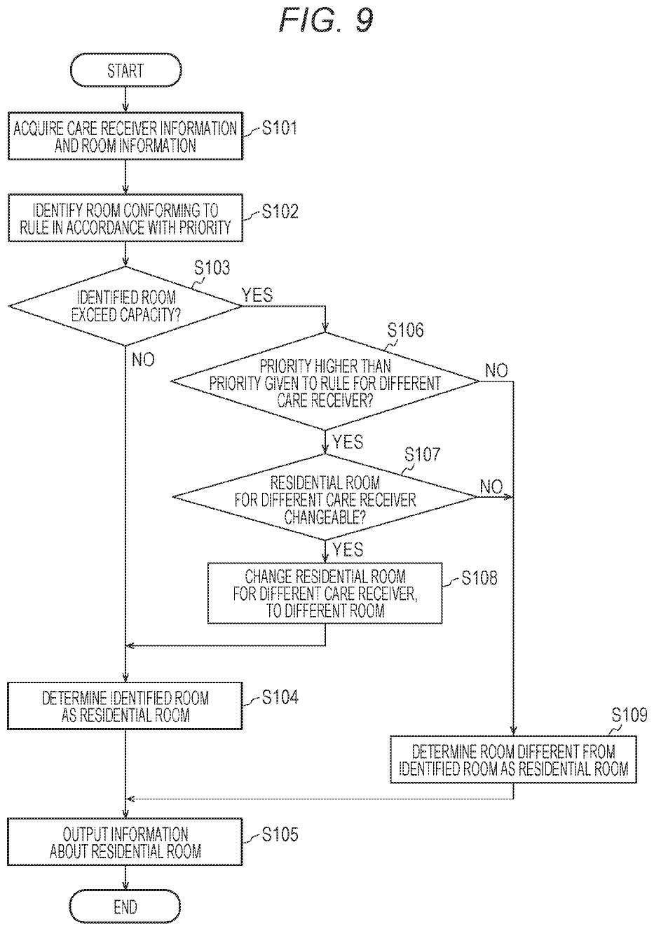

FIG. 9 is a flowchart illustrating procedures of processing executed by the information processing apparatus 200. The processing by the information processing apparatus 200 illustrated in the flowchart of FIG. 9 is stored in the form of a program in the storage 240 of the information processing apparatus 200, and is executed in such a manner that the CPU 210 controls the respective components.

In step S101, first, the information processing apparatus 200 acquires care receiver information and room information. For example, the information processing apparatus 200 acquires the care receiver information and the room information, in response to a user's instruction. The user inputs the instruction through, for example, the operation and display unit 260. The user inputs, for example, an instruction for preparation of a room list every predetermined time interval, such as every month. The room list is also referred to as a reservation list. The information processing apparatus 200 acquires the care receiver information and the room information from, for example, the storage 240.

In step S102, next, the information processing apparatus 200 associates the care receiver information and the room information, each acquired in the processing of step S101, with each other, based on the plurality of rules and their priorities each stored in, for example, the storage 240. In step S102, the information processing apparatus 200 then identifies a room conforming to one of the rules to which the higher priority is given. For example, the user inputs the necessity or unnecessity of application as to each of the plurality of rules and the priorities given to the rules, through the operation and display unit 260.

In step S103, next, the information processing apparatus 200 determines whether the room identified in the processing of step S102 exceeds its capacity. When the room identified in the processing of step S102 does not exceed its capacity, that is, when a result of the determination in step S103 is “NO”, the information processing apparatus 200 executes the following processing. In step S104, the information processing apparatus 200 determines that the room identified in the processing of step S102 is used as a residential room for the care receiver. In step S105, next, the information processing apparatus 200 outputs the information about the residential room for the care receiver determined in step S104, and then ends the processing. The capacity of each room is, for example, one.

On the other hand, when the room identified in the processing of step S102 exceeds its capacity, that is, when a result of the determination in step S103 is “YES”, the information processing apparatus 200 executes the following processing of step S106. In this processing, it is determined whether the priority given to the rule applied at the time when the room is identified is higher than the priority given to the rule for a different care receiver for which the relevant room is determined as the residential room. The priority given to the rule applied at the time when the relevant room is identified corresponds to the processing of step S102.

When the priority is equal to or lower than the priority given to the rule for the different care receiver, that is, when a result of the determination in step S106 is “NO”, the information processing apparatus 200 executes the following processing. In the processing of step S109, the information processing apparatus 200 determines that a room different from the room identified in the processing of step S102 is used as a residential room for the care receiver. Next, the information processing apparatus 200 executes the processing of step S105. In the processing of step S109, the information processing apparatus 200 identifies the different room, based on the plurality of rules and their priorities each stored in, for example, the storage 240. The information processing apparatus 200 determines that the different room thus identified is used as a residential room for the care receiver.

When the priority is higher than the priority given to the rule for the different care receiver, that is, when a result of the determination in step S106 is “YES”, the information processing apparatus 200 executes the following processing of step S107. In this processing, the information processing apparatus 200 determines whether the residential room for the different care receiver is changeable to a different room. When the residential room for the different care receiver is changeable to a different room, that is, when a result of the determination in step S107 is “YES”, the information processing apparatus 200 executes the following processing. In step S108, the information processing apparatus 200 changes the residential room for the different care receiver to the different room. The information processing apparatus 200 then executes the processing of step S104. When the residential room for the different care receiver cannot be changed to the different room, that is, when a result of the determination in step S107 is “NO”, the information processing apparatus 200 executes the processing of step S109. The processing of step S107 may be executed prior to the processing of step S106.

### <Operational Effects of Information Processing Apparatus 200>

The information processing apparatus 200 according to the present embodiment determines a residential room to be allocated to a care receiver scheduled to enter a care facility, from among a plurality of rooms in the care facility, based on care receiver information and room information. It is thus possible to allocate a suitable room as a residential room to each care receiver. It is therefore possible to lighten a burden on a staff of the care facility. The following describes the operational effects of the information processing apparatus 200.

Preparing a room list in consideration of situations of each care receiver scheduled to enter the care facility requires much time and effort and imposes a significant burden on the staff or the like of the care receiver. Preparing a room list for care receivers scheduled to enter the care facility for a short stay purpose particularly imposes a significant burden on the staff or the like of the care facility since staff or the like needs to prepare the room list frequently. In addition, preparing a room list by the staff or the like without taking situations of each care receiver into consideration may lead to deterioration in quality of care, trouble, and others.

In view of this, the information processing apparatus 200 determines a residential room to be allocated to a care receiver, based on care receiver information and room information. It is thus possible to allocate a suitable room as a residential room to each care receiver without a necessity of much time and effort to be expended by the staff of the care facility. It is therefore possible to lighten a burden on the staff of the like of the care facility and to enhance care services and others. It is also possible to inhibit deterioration in quality of care, occurrence of trouble of care receivers, and others, caused due to a room list. The information processing apparatus 200 is particularly usable as appropriate for preparation of a room list of care receivers scheduled to enter the care facility for a short stay purpose, the preparation imposing a significant burden on the staff.

The information processing apparatus 200 also determines a residential room to be allocated to a care receiver, based on a plurality of rules and their priorities, and therefore enables allocation of a more suitable room as a residential room to the care receiver. For example, applying a rule, “determine that a room used last time is used again as a residential room” allows a care receiver to utilize the care facility in an environment closer to the previous environment. Therefore, even a care receiver with symptoms of dementia is likely to utilize the care facility. In addition, even a care receiver with no symptoms of dementia is able to inhibit occurrence trouble. The term “trouble” used herein involves, for example, a situation in which a care receiver does not know where he/she is in the care facility or a situation in which a care receiver erroneously enters a wrong residential room. The information processing apparatus 200 is capable of further applying a rule, “determine that rooms in the same unit are used as residential rooms for care receivers equal in dementia level to each other”, a rule, “determine that rooms in the same unit are used as residential rooms for care receivers equal in assistance level to each other”, and the like. It is thus possible for care receivers to easily communicate with each other and to spend time in the care facility more comfortable. It is also possible for the staff or the like of the care facility to cut, for example, work to deliver equipment and the like for assistance.

The information processing apparatus 200 receives, from a user, the necessity or unnecessity of application as to each of a plurality of rules and priorities given to the rules; therefore, the rules and their priorities can be set freely for each user or for each care facility. It is therefore possible to determine a residential room for a care receiver, based on rules and their priorities set suitably and flexibly in accordance the situations and circumstances of the respective care facilities.

The information processing apparatus 200 is also capable of changing the determined residential room for the care receiver to a different room, in accordance with situations. It is thus possible to determine residential rooms suitable for a larger number of care receivers, irrespective of, for example, the order of application.

As described above, the information processing apparatus 200 according to the present embodiment determines a residential room to be allocated to a care receiver scheduled to enter a care facility, from among a plurality of rooms in the care facility, based on care receiver information and room information. It is thus possible to allocate a suitable room as a residential room to each care receiver. It is therefore possible to lighten a burden on a staff of the care facility.

The present invention has been described by way of the foregoing embodiment. However, the present invention is not limited to the foregoing embodiment only, and various modifications can be made within the scope of the claims.

In the exemplary description of the foregoing embodiment, for example, the information processing apparatus 200 determines a residential room to be allocated to a care receiver, based on a plurality of rules and their priorities. The information processing apparatus 200 may alternatively determine a residential room to be allocated to a care receiver, based on a single rule. The information processing apparatus 200 may alternatively determine a residential room to be allocated to a care receiver, using statistical processing, machine learning, or the like.

Also in the exemplary description of the foregoing embodiment, the information processing apparatus 200 functions as the acquisition unit 211, the accepting unit 212, the determination unit 213, the changing unit 214, and the output unit 215. However, the information processing apparatus does not necessarily have one or more functions among these functions. The information processing apparatus may alternatively have other functions. For example, the information processing apparatus 200 does not necessarily include the changing unit 214. The information processing apparatus 200 may alternatively have a function of canceling a residential room for a care receiver determined by the determination unit 213.

Also in the description of the foregoing embodiment, the information processing apparatus 200 is a specific example of a residential room determination apparatus according to the present invention. However, the user terminal 100 may serve as a residential room determination apparatus according to the present invention.

Also in the description of the foregoing embodiment, the information processing apparatus 200 acquires care receiver information and room information from the storage 240. However, the information processing apparatus 200 may acquire the care receiver information and the room information from, for example, another apparatus. For example, the information processing apparatus 200 may acquire the care receiver information from an external server of a care support office or the like.

Each of the user terminal 100 and the information processing apparatus 200 may further include one or more constituent elements in addition to the foregoing constituent elements. Alternatively, each of the user terminal 100 and the information processing apparatus 200 does not necessarily include one or more constituent elements among the foregoing constituent elements.

Each of the user terminal 100 and the information processing apparatus 200 may be practicable using a plurality of apparatuses. Alternatively, each of the user terminal 100 and the information processing apparatus 200 may be practicable using a single apparatus.

The function of each component may be implemented using another component. For example, the foregoing processing executed by the information processing apparatus 200 may be at least partially executed by another apparatus such as an application of the user terminal 100.

The processing described in the foregoing embodiment with reference to the flowchart is divided in accordance with main processing tasks for ease of the understanding of each processing task. However, the present invention is not restricted by how to divide the processing into processing steps. Each processing task is further dividable into more processing steps. Alternatively, one processing step may involve more processing tasks.

Means and methods for executing the various processing tasks in the system according to the foregoing embodiment are implementable using either a dedicated hardware circuit or a programmed computer. The foregoing program may be provided with, for example, a computer-readable recording medium such as a flexible disc or a compact disc (CD)-ROM. The foregoing program may be provided online via a network such as the Internet. In this case, the program recorded on the computer-readable recording medium is normally transferred to and stored in a storage unit such as a hard disc. The foregoing program may alternatively be provided as single application software. The foregoing program may be incorporated, as a function of a system, in software for an apparatus of the system.

Although embodiments of the present invention have been described and illustrated in detail, the disclosed embodiments are made for purpose of illustration and example only and not limitation. The scope of the present invention should be interpreted by terms of the appended claims.

What is claimed is:

1. A residential room determination apparatus comprising
a hardware processor that:
acquires care receiver information about a care receiver scheduled to enter a care facility, and room information about each of a plurality of rooms in the care facility; and
determines a residential room to be allocated to the care receiver from among the plurality of rooms, based on the care receiver information and the room information.

2. The residential room determination apparatus according to claim 1, wherein
the hardware processor identifies one of the rooms suitable for the care receiver in accordance with a rule that associates the care receiver information with the room information, and then determines the residential room.

3. The residential room determination apparatus according to claim 2, wherein
the hardware processor identifies the room in accordance with priorities given to a plurality of the rules.

4. The residential room determination apparatus according to claim 3, wherein
the hardware processor accepts, from a user, inputs as to the rules and the priorities given to the rules.

5. The residential room determination apparatus according to claim 4, wherein
the hardware processor accepts, from the user, inputs of necessity or unnecessity of application as to each of the rules previously stored in a storage unit, and the priorities given to the rules to be applied.

6. The residential room determination apparatus according to claim 3, wherein
the hardware processor identifies the room by applying the rules in descending order of the priorities.

7. The residential room determination apparatus according to claim 3, wherein
the hardware processor changes a first room determined as the residential room for the care receiver, to a second room different from the first room.

8. The residential room determination apparatus according to claim 7, wherein
the hardware processor, when identifying the first room for a care receiver different from the care receiver by applying one of the rules to which the higher priority is given, changes the residential room for the care receiver from the first room to the second room.

9. The residential room determination apparatus according to claim 7, wherein
the hardware processor changes the first room to the second room, based on an instruction from a user.

10. The residential room determination apparatus according to claim 1, wherein
the care receiver information includes at least one of basic information, information about a state of health, information about a state of care, or information about a history of entrance to the care facility, as to the care receiver.

11. The residential room determination apparatus according to claim 1, wherein
the room information includes at least one of identification information on each room, information about a location of each room, or information about a condition of a resident of each room.

12. The residential room determination apparatus according to claim 1, wherein
the hardware processor outputs information about the determined residential room for the care receiver.

13. The residential room determination apparatus according to claim 1, wherein
the care receiver is scheduled to enter the care facility for a short stay purpose.

14. A residential room determination method comprising
executing processing including:
acquiring care receiver information about a care receiver scheduled to enter a care facility, and room information about each of a plurality of rooms in the care facility; and
determining a residential room to be allocated to the care receiver from among the plurality of rooms, based on the care receiver information and the room information.

15. A non-transitory computer-readable recording medium storing a control program for causing a computer to execute the residential room determination method according to claim 14.
